(19) United States
(12) Patent Application Publication
(10) Pub. No.: US 2010/0185007 A1
(43) Pub. Date: Jul. 22, 2010

(54) PROCESS FOR PREPARING FLUOROAMIDE AND FLUORONITRILE

(76) Inventors: Akiyoshi Yamauchi, Settsu-shi (JP); Kensuke Mohara, Settsu-shi (JP); Keiko Takao, Settsu-shi (JP); Meiten Koh, Settsu-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 12/666,602

(22) PCT Filed: Jun. 24, 2008
(86) PCT No.: PCT/JP2008/061457

§ 371 Date: Dec. 23, 2009

(30) Foreign Application Priority Data
Jun. 25, 2007 (JP) 2007166526

(51) Int. Cl. C07C 253/20; C07C 231/02

(52) U.S. Cl. 558/313; 564/136

(57) ABSTRACT

There are provided simple processes for preparing a fluoroamide and a fluoronitrile which assure higher yield, i.e., a process for preparing a fluoroamide represented by the formula (2): CF₂═CF—R_{f}—CONH₂, wherein R_{f} is a perfluoroalkylene group or perfluorooxyalkylene group having 2 to 20 carbon atoms, by allowing a fluoroester represented by the formula (1): CF₂═CF—R_{f}—COOR, wherein R_{f} is as defined above; R is an alkyl group having 1 to 6 carbon atoms, to react with ammonia or ammonium hydroxide, and a process for preparing a fluoronitrile represented by the formula (3): CF₂═CF—R_{f}—CN, wherein R_{f} is as defined above, by allowing the fluoroamide obtained by the above-mentioned preparation process to react with a dehydrating agent (c) in a solvent (b) having an ether bond, an ester bond, a ketone group or a cyano group.

### TECHNICAL FIELD

The present invention relates to simple processes for preparing a fluoroamide and a fluoronitrile having olefin, and the processes assure high yield of the obtained fluoroamide and fluoronitrile.

### BACKGROUND ART

It is disclosed that a compound represented by the formula (3):

CF₂═CF—R_{f}—CN

wherein R_{f} is a perfluoroalkylene group or perfluorooxyalkylene group having 2 to 20 carbon atoms, is useful as a cure site monomer for a fluoroelastomer (for example, U.S. Pat. No. 3,467,638 and U.S. Pat. No. 4,281,092).

A process for preparing the above-mentioned conventional compound is such that (1) first, a corresponding starting ester or its derivative is reacted with ammonia gas at a low cooled temperature to synthesize an amide, and then (2) this amide is converted to a desired nitrile by a dehydration method.

In this conventional preparation process, since synthesis of an amide need be carried out at low temperature, it is difficult to undergo the reaction on a large scale, and there is a disadvantage that yield of a fluoronitrile is low. This disadvantage is caused by non-uniformity of a reaction medium and difficulty in separating a product. Since solubility of fluorinated compounds in usual organic solvents is generally very low, in these reactions, fluorinated solvents such as 1,1,2-trichloro-1,2,2-trifluoroethane (CFC-113) are frequently used as a solvent for extracting a product. CFC is now said to be undesirable from environmental point of view, and preparation thereof is decreasing gradually. Further, formation of a considerable amount of by-products arises due to sensitivity of olefin to a reagent (cf. U.S. Pat. No. 4,138,426). In this U.S. Pat. No. 4,138,426, yield of a reaction product in the first step of Example 8 conducted in ether (ethyl ether) is only 9%, and CFC-113 is used to separate a reaction product from a reactant. In the second step of U.S. Pat. No. 4,138,426, the amide obtained in the first step is dehydrated in a tetrahydrofuran solvent by using pyridine and trifluoroacetic acid anhydride as a dehydrating agent to synthesize a nitrile, and again separation of a product from a reactant is accelerated using CFC-113.

Disclosed in JP9-3027A is a process for preparing a fluoroamide and a fluoronitrile at yield higher than that of conventional synthesis processes by reacting fluoroester with ammonia or ammonium hydroxide in the absence of a solvent, in an oxygen-free solvent or in a solvent containing ether oxygen bonded to a perfluoroalkyl group. However, since the reaction of fluoroester with ammonia or ammonium hydroxide is carried out in the absence of a solvent, in an oxygen-free solvent or in a solvent containing ether oxygen bonded to a perfluoroalkyl group, in the case of using ammonia gas, the reaction temperature must be kept at −15° C. or low in order to inhibit liquidation of ammonia and generation of by-products, and even in the case of using ammonium hydroxide, since phase separation occurs, there are disadvantages such that a reaction speed is low and strong stirring is necessary. In this process, since a lot of by-products are generated, yield is up to about 80%.

Such being the case, it is desired that a more simple process for preparing a fluoroamide and a fluoronitrile at higher yield is developed.

### DISCLOSURE OF INVENTION

It is an object of the present invention to provide a process for preparing a fluoroamide and a process for preparing a fluoronitrile which assure higher yield and are simplified more as compared with conventional processes.

The present invention relates to a process for preparing a fluoroamide represented by the formula (2):

CF₂═CF—R_{f}—CONH₂ (2)

wherein R_{f} is a perfluoroalkylene group or perfluorooxyalkylene group having 2 to 20 carbon atoms, by allowing a fluoroester represented by the formula (1):

CF₂═CF—R_{f}—COOR (1)

wherein R_{f} is as defined above; R is an alkyl group having 1 to 6 carbon atoms, to react with ammonia or ammonium hydroxide, and the process is characterized in that the reaction is carried out in a solvent (a) having hydroxyl group.

It is preferable that the solvent (a) is alcohol.

It is preferable that the solvent having hydroxyl group, in which ammonia is dissolved, is added to the fluoroester.

It is preferable that R is methyl or ethyl.

It is preferable that R_{f} is

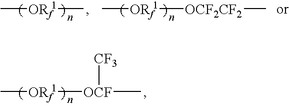

wherein —OR_{f}¹— is —OCF₂CF(CF₃)—,

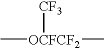

or —O(CF₂)ₘ— (m is an integer of 1 to 10); n is an integer of 1 to 5.

It is preferable that the above-mentioned —OR_{f}¹— is —OCF₂CF(CF₃)— and n is 1.

It is preferable that the above-mentioned —OR_{f}¹— is —O(CF₂)ₘ—, n is 1, and m is 2 to 5.

The present invention further relates to a process for preparing a fluoronitrile represented by the formula (3):

CF₂═CF—R_{f}—CN (3)

wherein R_{f} is as defined above, by allowing the fluoroamide obtained by the above-mentioned preparation process to react with a dehydrating agent (c),
and the process is characterized in that the reaction is carried out in a solvent (b) having an ether bond, an ester bond, a ketone group or a cyano group.

It is preferable that the dehydrating agent (c) is amine and acid anhydride.

It is preferable that the amine is added dropwise to a mixture of the fluoroamide and the acid anhydride so that a molar ratio of the amine to the acid anhydride is 0.8 to 3.0:1.0.

It is preferable that the amine is pyridine or triethylamine.

It is preferable that the acid anhydride is trifluoroacetic acid anhydride.

It is preferable that the amine is pyridine or triethylamine and the acid anhydride is trifluoroacetic acid anhydride.

It is preferable that the fluoroamide is a crude fluoroamide which has not been refined.

### BEST MODE FOR CARRYING OUT THE INVENTION

The present invention relates to the process for preparing a fluoroamide by allowing a fluoroester to react with ammonia or ammonium hydroxide.

In the present invention, the fluoroester can be represented by the formula (1):

CF₂═CF—R_{f}—COOR (1)

wherein R_{f} is a perfluoroalkylene group or perfluorooxyalkylene group having 2 to 20 carbon atoms; R is an alkyl group having 1 to 6 carbon atoms.

In the formula (1), R_{f} is a perfluoroalkylene group or perfluorooxyalkylene group having 2 to 20 carbon atoms. When R_{f} is a perfluoroalkylene group or perfluorooxyalkylene group having one carbon atom, there is a tendency that the fluoroester itself is unstable and is easily decomposed under the reaction conditions, thereby decreasing yield significantly, and when R_{f} is a perfluoroalkylene group or perfluorooxyalkylene group having more than 20 carbon atoms, there is a tendency that since a reaction solvent which can solve the fluoroester is little, phase separation occurs, resulting in decreased reaction efficiency. Example of R_{f} is

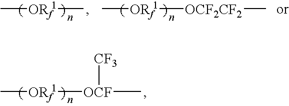

wherein —OR_{f}¹— is —OCF₂CF(CF₃)—,

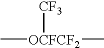

or —O(CF₂)ₘ— (m is an integer of 1 to 10); n is an integer of 1 to 5. When n is an integer exceeding 5, there is a tendency that since a reaction solvent which can solve the fluoroester is little, phase separation occurs, resulting in decreased reaction efficiency.

When —OR_{f}¹— is —O(CF₂)ₘ—, m is preferably an integer of 1 to 10, more preferably an integer of 2 to 5. When m is an integer exceeding 10, there is a tendency that since a reaction solvent which can solve the fluoroester is little, phase separation occurs, resulting in decreased reaction efficiency.

In the formula (1), R is an alkyl group having 1 to 6 carbon atoms, preferably methyl or ethyl. When R is an alkyl group having more than six carbon atoms, there is a tendency that reaction efficiency at amidation is low, formed alcohol is hardly removed and it is difficult to carry out refining.

With respect to such a fluoroester or a precursor acid to be easily subjected to esterification, known ones can be used (cf. U.S. Pat. No. 3,546,186, U.S. Pat. No. 4,138,486, U.S. Pat. No. 4,275,226, U.S. Pat. No. 4,281,092, and Zh. Org. Kim. 16, 540 (1980)).

In the process for preparing a fluoroamide of the present invention, the reaction of the fluoroester with ammonia or ammonium hydroxide is carried out in the solvent (a) having hydroxyl group. Since ammonia is easily added to a vinyl group of the fluoroester of the present invention, a reaction temperature need be low, which leads to decrease in productivity. It is preferable that alcohol is used as the solvent (a) in that an effect of inhibiting side reaction due to ammonia, namely, addition of ammonia to a vinyl group of the fluoroester represented by the formula (1) is high, and the reaction temperature can be high.

Alcohols may be either compounds having single hydroxyl group such as monool or compounds having plural hydroxyl groups such as diol and triol.

Examples of alcohols suitable for the present invention are methanol, ethanol, ethylene glycol, butanol, butanetriol, propanol and the like.

Among these, alcohols having 1 to 6 carbon atoms are more preferred, and methanol, ethanol, propanol and butanol are further preferred in that a boiling point is low, removal by post-treatment is easy and refining is easy.

In addition, alcohols having a halogen-substituted alkyl group or an ether group may be used as the solvent (a). Among these alcohols, preferred are alcohols having a fluorine-substituted alkyl group or an ether group.

Specifically, CF₃CH₂OH, CF₃CF₂CH₂OH, HCF₂CF₂CH₂OH, CF₃CFHCF₂CH₂OH, (CF₃)₂CHOH, CF₃CF₂CF₂OCF(CF₃)CH₂OH and the like are suitably used as alcohols having a fluorine-substituted alkyl group and an ether group. Alcohols may be used alone or may be used in a mixture of two or more thereof.

Also, ammonia to be used on the process for preparing a fluoroamide of the present invention may be (A-1) added in the gaseous form directly to the solvent and reacted with the fluoroester or (A-2) dissolved previously in water to be ammonium hydroxide which is then added to the solvent and reacted with the fluoroester. Or, (A-3) ammonia may be dissolved previously in the solvent (a) and then reacted with the fluoroester.

The use of water has an adverse effect on the following step, and in order to make refining easy, it is more preferable to employ the method (A-1) or (A-3).

In the preparation process of the present invention, a molar ratio of ammonia or ammonium hydroxide to 1.0 of a fluoroester is preferably 1.0 to 2.5, more preferably 1.1 to 2.0. When the molar ratio of ammonia or ammonium hydroxide to 1.0 of a fluoroester is less than 1.0, yield tends to be lowered because a fluoroester remains un-reacted and after the following step, separation at the refining is difficult, and when the molar ratio exceeds 2.5, by-products generated by addition of un-reacted ammonia to a vinyl group of the fluoroester represented by the formula (1) tends to increase, thereby lowering yield.

The reaction of the fluoroester with ammonia is carried out preferably at −25° C. to 50° C., more preferably at −5° C. to 40° C. When the reaction of the fluoroester with ammonia is carried out at a temperature lower than −25° C., the reaction tends to proceed slowly, and when the reaction is carried out at a temperature exceeding 50° C., by-products generated by addition of ammonia to a vinyl group of the fluoroester represented by the formula (1) tends to increase. The reaction of the fluoroester with ammonium hydroxide is carried out preferably at −25° C. to 30° C., more preferably at −10° C. to 20° C. When the reaction of the fluoroester with ammonium hydroxide is carried out at a temperature lower than −25° C., the reaction tends to proceed slowly, and when the reaction is carried out at a temperature exceeding 30° C., by-products generated by addition of ammonium hydroxide to a vinyl group of the fluoroester represented by the formula (1) tends to increase.

In the preparation process of the present invention, by carrying out the reaction of the fluoroester with ammonia or ammonium hydroxide in the solvent (a), the fluoroamide can be easily separated from a reaction mixture and the fluoroamide having a very high purity can be obtained by distillation. According to the preparation process of the present invention, yield of the fluoroamide represented by the formula (2) is high. Yield converted to mole is more than 80%, and is usually higher than 85%.

The fluoronitrile of the present invention represented by the formula (3):

CF₂═CF—R_{f}—CN (3)

wherein R_{f} is as defined above, is prepared by allowing the fluoroamide represented by the formula (2) to react with the dehydrating agent (c) in the solvent (b).

Examples of the solvent (b) are solvents having an ether bond, an ester bond, a ketone group or a cyano group.

Examples of the solvent having an ether bond are tetrahydrofuran (THF), 1,4-dioxane, diglyme, triglyme, tetraglyme and the like. Here, glyme is a general term for symmetric glycol diethers.

Examples of the solvent having an ester bond are methyl acetate, ethyl acetate, butyl acetate, methyl propionate, ethyl propionate and the like.

Examples of the solvent having a ketone group are acetone, methyl ethyl ketone, methyl isobutyl ketone and the like.

Examples of the solvent having a cyano group are acetonitrile, propionitrile, benzonitrile and the like.

Among the solvents (b), water soluble solvents having a low boiling point such as tetrahydrofuran, acetone and acetonitrile are preferred from the viewpoint of good compatibility with the fluoroamide and the dehydrating agent (c) and easy refining after the reaction.

Examples of the dehydrating agent (c) are amines such as pyridine and triethylamine, acid anhydrides such as trifluoroacetic acid anhydride, acetic anhydride and maleic anhydride, phosphoric acid, phosphorus pentoxide, phosphorus pentachloride, phosphorus triphenyl, phosgene, fluorophosgene and the like. Among the dehydrating agents (c), the above-mentioned amines and acid anhydrides are preferred from the viewpoint that the vinyl group of the fluoroamide represented by the formula (2) has low stability against acid and heat, heating is not necessary, and a solution hardly becomes acidic.

In the process for preparing the fluoronitrile of the present invention, a molar ratio of the dehydrating agents (c) to 1.0 of the fluoroamide is preferably 0.9 to 2.5, more preferably 1.1 to 2.0. When the molar ratio of the dehydrating agents (c) to 1.0 of the fluoroamide is less than 0.9, there is a tendency that a lot of unreacted fluoroamide remains and yield is lowered, and when the molar ratio exceeds 2.5, there is a tendency that cost becomes high due to the presence of a lot of un-reacted dehydrating agent and separation at refining becomes difficult, thereby lowering yield.

When amine and acid anhydride are used as the dehydrating agent (c), dehydration reaction does not occur only by one of them, and the dehydration reaction occurs only by activation of acid anhydride with amine.

When using acid anhydride and amine as the dehydrating agent (c), the molar ratio of the acid anhydride to 1.0 of the fluoroamide is preferably 1.0 to 2.5, more preferably 1.1 to 1.6. When the molar ratio of the acid anhydride to 1.0 of the fluoroamide is less than 1.0, there is a tendency that the fluoroamide cannot be completely converted by the reaction, and when the molar ratio of the acid anhydride exceeds 2.5, the reaction can be advanced completely, but there is a tendency to hardly remove by washing with water.

When using the acid anhydride and the amine as the dehydrating agent (c), there is a method of (B-1) adding the acid anhydride dropwise to a mixture of the fluoroamide and the amine in the presence of the solvent (b) or (B-2) adding the amine dropwise to a mixture of the fluoroamide and the acid anhydride in the presence of the solvent (b). Any of the methods (B-1) and (B-2) may be used, but the fluoroamide tends to be easily decomposed in a basic solvent, it is preferable to employ the method (B-2).

When employing the method (B-1), the molar ratio of the amine to 1.0 of acid anhydride is preferably 1.5 to 3.0, more preferably 1.5 to 2.0. When the molar ratio of the amine to 1.0 of acid anhydride is less than 1.5, there is a tendency that the fluoroamide cannot be completely converted by the reaction, and when the molar ratio of the amine exceeds 3.0, a lot of by-products tend to be generated.

In the method (B-1), the reaction is carried out preferably at −30° C. to 5° C., more preferably at −25° C. to 0° C. In the method (B-1), when the reaction temperature is lower than −30° C., there is a tendency that the reaction speed is slow and productivity is low, and when the reaction temperature exceeds 5° C., there is a tendency that an amount of by-products resulting from a reaction of vinyl group of the fluoroamide is increased and yield is lowered.

When employing the method (B-2), the molar ratio of the amine to 1.0 of the acid anhydride is preferably 0.8 to 2.5, more preferably 1.0 to 2.0. When the molar ratio of the amine to 1.0 of the acid anhydride is less than 0.8, there is a tendency that the fluoroamide cannot be completely converted by the reaction, and when the molar ratio of the amine exceeds 2.5, a lot of by-products tend to be generated.

In the method (B-2), the reaction is carried out preferably at −30° C. to 50° C., more preferably at −5° C. to 40° C. In the method (B-2), when the reaction temperature is lower than −30° C., there is a tendency that the reaction speed is slow and productivity is low, and when the reaction temperature exceeds 50° C., there is a tendency that an amount of by-products resulting from a reaction of vinyl group of the fluoroamide is increased and yield is lowered.

The method (B-2) differs from the method (B-1) in that in the former method, basicity of the solvent can be inhibited, and thus, in the method (B-2), the reaction temperature can be increased by allowing the fluoroamide being weak to a base to be hardly decomposed. Therefore, the method (B-2) is preferred since the reaction speed can be improved even in the case of reaction on a large scale where a heat removing efficiency is lowered.

Aprotic amines such as pyridine and triethylamine are preferred as the amine to be used in the above-mentioned (B-1) and (B-2), and carboxylic acid anhydrides such as acetic anhydride, maleic anhydride and trifluoroacetic acid anhydride are preferred as the acid anhydride.

In the preparation process of the present invention, by carrying out the reaction of the fluoroamide with the dehydrating agent (c) in the solvent (b), most of the fluoronitrile is separated from the reaction product in the form of well-defined liquid phase, and another phase is a solvent phase containing a reaction residue of the dehydrating agent. The liquid phase containing the fluoronitrile is separated, washed with water and subsequently distilled, thereby enabling the fluoronitrile to be obtained at high yield. According to the preparation process of the present invention, yield of the fluoronitrile represented by the formula (3) is high, and usually exceeds 80% in mole percent, and yield higher than 85% is often achieved.

In the preparation processes of the present invention, the process for preparing the fluoroamide by allowing the fluoroester represented by the formula (1) to react with ammonia or ammonium hydroxide in the solvent (a) and the process for preparing the fluoronitrile by allowing the fluoroamide represented by the formula (2) to react with the dehydrating agent (c) in the solvent (b) are processes employed for preparing a fluoronitrile from a fluoroester. In these processes for preparing the fluoronitrile from the fluoroester, the fluoroamide which is an intermediate may be separated and refined.

Also, in the preparation process of the present invention, the steps for preparing the fluoronitrile from the fluoroester can be carried out continuously without refining of the fluoroamide which is an intermediate. For example, in the case of carrying out these steps continuously, it is possible to allow the fluoroester to react with ammonia or ammonium hydroxide in the solvent (a) and remove excessive ammonia and by-products, i.e. methanol under reduced pressure, and distillation of crude fluoroamide which requires heavy cost and takes time is not necessary. Then, by allowing the fluoroamide to react with the dehydrating agent (c) in the solvent (b), the fluoronitrile represented by the formula (3) can be prepared. The obtained fluoronitrile is, as mentioned above, refined by separating, washing with water and distilling.

The reaction steps to be carried out continuously have a significant advantage that a step for refining the fluoroamide can be eliminated. Further, in the preparation process of the present invention, cooling of ammonia or ammonium hydroxide to form it into a liquid is not necessary, the fluoroamide and the fluoronitrile can be simply produced, and increase in production scale is easy.

### EXAMPLE

The present invention is then explained by means of examples, but is not limited to these examples.

Measuring methods employed in the present invention are as follows.

### (Measurement of Fluoroamide)

NMR: AC-300 available from BRUKER is used.

¹⁹F-NMR: (acetone): −81.10 ppm (3F), −83.33 to −84.64 ppm (2F), −85.79 ppm (2F), −114.31 to −114.97 ppm (1F), −122.44 to −123.26 ppm (3F), −137.11 to −137.85 ppm (1F), −146.20 ppm (1F)

Measuring condition: 300 MHz (tetramethylsilane=0 ppm)

GC: GC-17A available from Shimadzu Corporation is used. A column of DB624 (length: 60 m, inner diameter: 0.32 mm, film thickness: 1.8 μm) is used.

Measurement is carried out by holding 0 min at 70° C., heating up to 230° C. at a temperature elevating rate of 10° C./min and holding for 14 minutes. Fluoroamide is detected after a lapse of 13.33 min.

### (Measurement of Fluoronitrile)

NMR: AC-300 available from BRUKER is used.

¹⁹F-NMR: (acetone): −81.13 ppm (3F), −84.86 to −86.32 ppm (4F), −109.69 ppm (2F), −114.03 to −114.62 ppm (1F), −122.24 to −123.01 ppm (1F), −137.30 to −138.03 ppm (1F), −145.62 ppm (1F)

Measuring condition: 282 MHz (trichlorofluoromethane=0 ppm)

GC: GC-17A available from Shimadzu Corporation is used. A column of DB624 (length: 60 m, inner diameter: 0.32 mm, film thickness: 1.8 μm) is used.

Measurement is carried out by holding 0 min at 70° C., heating up to 230° C. at a temperature elevating rate of 10° C./min and holding for 14 minutes. Fluoronitrile is detected after a lapse of 4.26 min.

### Synthesis Example 1

Into a 100 liter reactor were poured 61.89 kg of fluoroester represented by the following formula:

CF₂═CFO—CF₂CF(CF₃)—O—CF₂CF₂COOCH₃

and 20 kg of methanol, and after replacing the inside of the reactor with nitrogen gas, 22 liter of a solution of ammonia-methanol of 7 mole/liter was added dropwise with stirring at 20° C. After completion of the addition, stirring was carried out for another one hour. After completion of the reaction, purity by GC after distilling off of methanol was 99.2%. After completion of the reaction, distillation of methanol and ammonia was carried out, and 58.55 kg of fluoroamide having purity by GC of 99.2% and represented by:

CF₂═CFO—CF₂CF(CF₃)—O—CF₂CF₂CONH₂

was obtained (yield: 98.1%).

To the fluoroamide obtained above were added 20 liter of THF and 29.5 kg of pyridine, and after replacing the inside of the reactor with nitrogen gas, 39.3 kg of trifluoroacetic acid anhydride was added dropwise with stirring at −5° C. After completion of the addition, stirring was continued for another 0.5 hour. After completion of the reaction, the solution was separated with water and the lower organic layer was taken out to make an analysis by GC. According to GC analysis, 53.7 kg of fluoronitrile having purity by GC of 98.1% and represented by the following formula:

CF₂═CFO—CF₂CF(CF₃)—O—CF₂CF₂CN

was obtained (yield: 96%).

The obtained crude fluoronitrile was subjected to rectification with a 4-staged rectifier, and 51.8 kg of a refined fluoronitrile having purity by GC of 99.8% was obtained (yield: 96%).

### Synthesis Example 2

Into a 2-liter reactor were poured 555.00 g of the fluoroamide prepared in the same manner as in Synthesis Example 1 and represented by the following formula:

CF₂═CFO—CF₂CF(CF₃)—O—CF₂CF₂CONH₂,

450 ml of THF and 352.44 g of trifluoroacetic acid anhydride, and after replacing the inside of the reactor with nitrogen gas, 271.65 g of pyridine was added dropwise at 20° C. with stirring. After completion of the addition, stirring was continued for another 0.5 hour. After completion of the reaction, the solution was separated with water and the lower organic layer was taken out to make an analysis by GC. According to GC analysis, 525.1 g of fluoronitrile having purity by GC of 88.3% and represented by the following formula:

CF₂═CFO—CF₂CF(CF₃)—O—CF₂CF₂CN

was obtained (yield: 92%).

The obtained crude fluoronitrile was subjected to rectification with a 5-staged rectifier, and 495.2 g of a refined fluoronitrile having purity by GC of 99.3% was obtained (yield: 86.8%).

### Comparative Example 1

Into a 100 ml four-necked flask was poured 50 g of fluoroester represented by the following formula:

CF₂═CFO—CF₂CF(CF₃)—O—CF₂CF₂COOCH₃

and after replacing the inside of the reactor with nitrogen gas, 1.89 g of ammonia gas was introduced at 20° C. by bubbling while stirring. After completion of the introduction, 1-hour stirring was carried out.

According to GC analysis, fluoroester disappeared, but the target product, fluoroamide represented by the following formula:

CF₂═CFO—CF₂CF(CF₃)—O—CF₂CF₂CONH₂

was not generated and only by-product was generated.

### Synthesis Example 3

Into a 100 ml four-necked flask were poured 11.71 g of the fluoroamide prepared in the same manner as in Synthesis Example 1 and represented by the following formula:

CF₂═CFO—CF₂CF(CF₃)—O—CF₂CF₂CONH₂,

30 ml of THF and 5.9 g of pyridine, and after replacing the inside of the flask with nitrogen gas, 7.86 g of trifluoroacetic acid anhydride was added dropwise at 20° C. with stirring. After completion of the addition, stirring was continued for another 0.5 hour. After completion of the reaction, the solution was separated with water and the lower organic layer was taken out to make an analysis by GC. As a result, only fluoronitrile having purity by GC of 20% and represented by the following formula:

CF₂═CFO—CF₂CF(CF₃)—O—CF₂CF₂CN

was generated, and many peaks of by-products were confirmed.

### Synthesis Example 4

Into a 100 liter reactor were poured 61.74 kg of fluoroester represented by the following formula:

CF₂═CFO—(CF₂)₅COOCH₂CH₃

and 20 kg of ethanol, and after replacing the inside of the reactor with nitrogen gas, 22 liter of a solution of ammonia ethanol of 7 mole/liter was added dropwise at 20° C. with stirring. After completion of the addition, stirring was carried out for another one hour. After completion of the reaction, purity by GC after distilling off of ethanol was 99.3%. After completion of the reaction, distillation of ethanol and ammonia was carried out under reduced pressure, and 56.50 kg of fluoroamide having purity by GC of 99.3% and represented by:

CF₂═CFO—(CF₂)₅—CONH₂

was obtained (yield: 98.3%).

To the fluoroamide obtained above were added 20 liter of THF and 30.0 kg of pyridine, and after replacing the inside of the reactor with nitrogen gas, 39.58 kg of trifluoroacetic acid anhydride was added dropwise at −5° C. with stirring. After completion of the addition, stirring was continued for another 0.5 hour. After completion of the reaction, the solution was separated with water and the lower organic layer was taken out to make an analysis by GC. According to GC analysis, 51.92 kg of fluoronitrile having purity by GC of 98.3% and represented by the following formula:

CF₂═CFO—(CF₂)₅—CN

was obtained (yield: 96%).

The obtained crude fluoronitrile was subjected to rectification with a 4-staged rectifier, and 51.3 kg of a refined fluoronitrile having purity by GC of 99.8% was obtained (yield: 95%).

### SYNTHESIS EXAMPLES 5 to 9

A solvent and amine were added to fluoroamide:

CF₂═CFO—CF₂CF(CF₃)—O—CF₂CF₂CONH₂,

and after replacement with nitrogen, acid anhydride was added dropwise at room temperature with stirring. After completion of the addition, 0.5-hour stirring was continued at a specified reaction temperature. After completion of the reaction, the occurrence of the reaction was confirmed by GC analysis. Kind and amounts of the solvent, amine and acid anhydride, reaction temperature and results of the reaction are shown in Table 1.

TABLE 1

| | Synthesis Example | | | | |
|---|---|---|---|---|---|
| | 5 | 6 | 7 | 8 | 9 |
| Solvent | Ethyl acetate | Acetonitrile | THF | THF | THF |
| (Amount (L)) | (2) | (2) | (2) | (2) | (2) |
| Amine | Pyridine | Pyridine | Triethylamine | Pyridine | Pyridine |
| (Amount (kg)) | (2.95) | (2.95) | (3.77) | (2.95) | (2.95) |
| Acid anhydride | Trifluoroacetic | Trifluoroacetic | Trifluoroacetic | Acetic | Maleic |
| (Amount (kg)) | acid anhydride | acid anhydride | acid anhydride | anhydride | anhydride |
| | (3.93) | (3.93) | (3.93) | (1.91) | (1.87) |
| Reaction temperature | −5 | −5 | −5 | under refluxing | under refluxing |
| (° C.) | | | | | |
| Conversion ratio | not less than | not less than | not less than | 80% | 80% |
| | 99% | 99% | 99% | | |

### INDUSTRIAL APPLICABILITY

In the present invention, fluoroamide can be obtained at high yield by allowing a fluoroester to react with ammonia or ammonium hydroxide in a solvent (a) having hydroxyl group. Further, since neither a step for forming ammonia into a liquid nor a step for cooling for inhibiting generation of by-products is required, the reaction can be carried out simply and production scale can be increased easily. Also, according to the preparation process of the present invention, since it is not necessary to extract the fluoroamide from the reaction medium by using a perhalogenated solvent, no environmental problem occurs. Further, in the preparation process of the present invention, since the reaction of the fluoroamide with the dehydrating agent (c) is carried out in a solvent (b) having an ether bond, an ester bond, a ketone group or a cyano group, the reaction product can be obtained in a solvent phase and fluoronitrile phase, and the fluoronitrile can be easily separated from such phases even without using a halogenated solvent. Also, side reaction is inhibited, and high yield can be achieved even without making a reaction temperature low.

Further, according to the preparation process of the present invention, preparation of fluoronitrile through preparation of fluoroamide from fluoroester can be carried out continuously without refining of the fluoroamide, which is economical from the viewpoint of time and cost.

1. A process for preparing a fluoroamide represented by the formula (2):
CF₂═CF—R_{f}—CONH₂ (2)
wherein R_{f} is a perfluoroalkylene group or perfluorooxyalkylene group having 2 to 20 carbon atoms, by allowing a fluoroester represented by the formula (1):
CF₂═CF—R_{f}—COOR (1)
wherein R_{f} is as defined above; R is an alkyl group having 1 to 6 carbon atoms, to react with ammonia or ammonium hydroxide,
said process is characterized in that the reaction is carried out in a solvent (a) having hydroxyl group.

2. The process for preparing a fluoroamide of claim 1, wherein the solvent (a) is alcohol.

3. The preparation process of claim 1, wherein a solvent having hydroxyl group, in which ammonia is dissolved, is added to the fluoroester.

4. The process for preparing a fluoroamide of claim 1, wherein R is methyl or ethyl.

5. The process for preparing a fluoroamide of claim 1, wherein R_{f} is 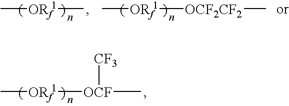
wherein —OR_{f}¹— is —OCF₂CF(CF₃)—, 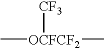
or —O(CF₂)ₘ— (m is an integer of 1 to 10); n is an integer of 1 to 5.

6. The process for preparing a fluoroamide of claim 5, wherein —OR_{f}¹— is —OCF₂CF(CF₃)— and n is 1.

7. The process for preparing a fluoroamide of claim 5, wherein —OR_{f}¹— is —O(CF₂)ₘ—, n is 1, and m is 2 to 5.

8. A process for preparing a fluoronitrile represented by the formula (3):
CF₂═CF—R_{f}—CN (3)
wherein R_{f} is as defined above, by allowing the fluoroamide obtained by the preparation process of claim 1 to react with a dehydrating agent (c),
said process is characterized in that the reaction is carried out in a solvent (b) having an ether bond, an ester bond, a ketone group or a cyano group.

9. The process for preparing a fluoronitrile of claim 8, wherein the dehydrating agent (c) is amine and acid anhydride.

10. The process for preparing a fluoronitrile of claim 9, wherein the amine is added dropwise to a mixture of the fluoroamide and the acid anhydride in the presence of the solvent (b).

11. The process for preparing a fluoronitrile of claim 9, wherein a molar ratio of the amine to 1.0 of the acid anhydride is 0.8 to 3.0.

12. The process for preparing a fluoronitrile of claim 9, wherein the amine is pyridine or triethylamine.

13. The process for preparing a fluoronitrile of claim 9, wherein the acid anhydride is trifluoroacetic acid anhydride.

14. The process for preparing a fluoronitrile of claim 8, wherein the fluoroamide is a crude fluoroamide which has not been refined.
